(19) United States
(12) Patent Application Publication
(10) Pub. No.: US 2023/0256629 A1
(43) Pub. Date: Aug. 17, 2023

(54) ELECTRIC CUTTER ASSEMBLY

(71) Applicant: X.J. Electrics (Hubei) Co., Ltd., Huanggang City (CN)

(72) Inventor: Yun PAN, Huanggang City (CN)

(73) Assignee: X.J. Electrics (Hubei) Co., Ltd., Huanggang City (CN)

(21) Appl. No.: 17/669,607

(22) Filed: Feb. 11, 2022

(51) Int. Cl. B26B 7/00; H02J 7/00; A61L 2/10

(52) U.S. Cl. CPC B26B 7/00; H02J 7/0042; A61L 2/10; A61L 2202/16

(57) ABSTRACT

The present application discloses an electric cutter assembly, including a handle component and a blade component configured movably on the handle component, wherein the handle component is provided therein with a driving component for driving the blade component to reciprocate relative to the handle component and a storage battery electrically connected to the driving component, and the handle component is further provided with a charging interface electrically connected to the storage battery. The electric cutter assembly provided in the present application has the driving component powered with a built-in storage battery so that the handle component can drive the blade component to reciprocate and cut food materials without connecting to an external power source, which effectively avoids the hindrance of the power cord in the use of the electric cutter assembly, and the user can move the electric cutter assembly to any desired position to perform food material processing, which is convenient for the user.

### TECHNICAL FIELD

The present application relates to the technical field of cutters, more particularly, to an electric cutter assembly.

### BACKGROUND ART

A conventional cutter generally includes a handle and a blade that are connected, and a user may hold the handle, cut and chop food materials with the blade. It's labor-intensive to use the conventional cutter, and the user is easy to get exhausted.

As a result, electric cutters appeared on the market. The electric cutters in the prior art generally use a driving mechanism provided in the handle to drive the blade to move reciprocally relative to the handle so that a user can perform operations such as cutting the food materials by merely holding the handle and letting the blade move. However, connecting the electric cutter to a power source to enable the blade to move continuously requires a power cord that connects the electric cutter to the power source, but the power cord may hinder the user in operating the electric cutter to process the food materials, and the user has to operate the electric cutter in a limited range around the power source due to a limited length of the power cord, leading to a poor user experience.

### SUMMARY OF THE INVENTION

### Technical Problem

It is an object of the present application to provide an easy-to-use electric cutter assembly.

### Technical Solution

To solve the above-mentioned technical problem, the present application adopts the following technical solution: an electric cutter assembly, including a handle component and a blade component configured movably on the handle component, wherein the handle component is provided therein with a driving component for driving the blade component to reciprocate relative to the handle component and a storage battery electrically connected to the driving component, and the handle component is further provided with a charging interface electrically connected to the storage battery.

In an embodiment, the electric cutter assembly further includes a charging cord adapted to the charging interface.

In an embodiment, the electric cutter assembly further includes a base having a second chamber for receiving the handle component, the second chamber having a charging plug for mating with the charging interface.

In an embodiment, the electric cutter assembly further includes a sheath component having at least one removable blade component received therein, the base having a first chamber for receiving the sheath component.

In an embodiment, the sheath component has a first housing for receiving the blade component, a through hole is provided in a bottom surface of the first housing to penetrate the first housing, a UV lamp is provided in the first chamber, and light emitted from the UV lamp is directed to the through hole.

In an embodiment, a movable triggering lever is provided on the base and an end of the triggering lever extends into the first chamber, a second switch electrically connected to the UV lamp is further provided on the base, and the second switch is configured near an end of the triggering lever away from the first chamber.

In an embodiment, the second switch includes a shrapnel that abuts against the end of the triggering lever away from the first chamber.

In an embodiment, the first housing is provided therein with a fixed seat for receiving the blade component and a movable seat pressing the blade component against the fixed seat, the movable seat is provided with a movable button for driving the movable seat to move, and the movable button protrudes from the first housing.

In an embodiment, the movable seat has a movable plate to which the movable button is mounted, an elastic member for driving the movable plate to move away from the fixed seat is provided between the movable plate and the fixed seat, and a clasp is provided on the fixed seat to mate with the movable plate.

In an embodiment, an engaging block is provided on the movable plate, and the engaging block is inserted into the fixed seat and moved towards the fixed seat by the elastic member.

In an embodiment, the handle component has a second housing, and a bracket is provided in the second housing, the bracket is provided with a safety component, a switch component, a locking component, and a driving component, the driving component includes a mount movable relative to the bracket and a driving member for driving the mount to reciprocate, and the mount mates with the blade component; the locking component includes a rotatable clamping portion for pressing the blade component against the mount, and the locking component further includes an unlocking button to drive the clamping portion to rotate in a direction away from the mount; the switch component includes a trigger configured rotatably on the bracket, and the trigger has a limiting portion for driving the clamping portion to rotate in a direction closer to the mount; the safety component has a first locking portion that abuts against the limiting portion to limit rotation of the trigger.

In an embodiment, the driving component further includes a first switch electrically connected to the driving member, the trigger has a triggering portion that mates with the first switch, and the limiting portion abuts against the clamping portion when the triggering portion contacts the first switch.

In an embodiment, a second locking portion abutting against the triggering portion is also provided on the safety component, and the trigger is provided with a locking notch for receiving the second locking portion.

In an embodiment, the mount has a receiving groove for receiving a portion of the blade component, the clamping portion extending into the receiving groove for abutting against the blade component.

In an embodiment, the blade component includes two blades arranged side by side, two mounts are provided, and the two mounts are arranged side by side, surfaces of the two mounts away from each other are provided with the receiving grooves, respectively, and one of the receiving grooves receives one of the blades.

In an embodiment, a locking shrapnel is provided within the receiving groove to abut against the blade and press the blade against the mount.

In an embodiment, the blade is provided with a protruding limiting post, and a limiting hole mating with the limiting post is provided in the locking shrapnel.

In an embodiment, one end of the mount close to the driving member has an oblong hole, an output end of the driving member is provided with a transmission gear, a protruding transfer link is provided on a sidewall of the transmission gear, the transfer link is eccentrical with respect to a rotational axis of the transmission gear, and the transfer link extends into the oblong hole and abuts against an inner peripheral wall of the oblong hole.

In an embodiment, an end of the unlocking button close to the clamping portion has a first inclined surface, and the clamping portion is provided with a second inclined surface mating with the first inclined surface.

In an embodiment, a side of the limiting portion close to the clamping portion has a third inclined surface, and the clamping portion is provided with a fourth inclined surface mating with the third inclined surface.

The present application is advantageous because the electric cutter assembly provided in the present application has the driving component powered with a built-in storage battery so that the handle component can drive the blade component to reciprocate and cut food materials without connecting to an external power source, which effectively avoids the hindrance of the power cord in the use of the electric cutter assembly, and the user can move the electric cutter assembly to any desired position to perform food material processing, having only to charge the storage battery when the electric cutter assembly is left unused, which is convenient for the user.

### BRIEF DESCRIPTION OF THE DRAWINGS

The accompanying drawings, which are included to provide a further understanding of the present application and are incorporated herein, illustrate embodiment(s) of the present application and together with the description serve to explain the present application, instead of an undue limitation of the present application.

To illustrate the technical solution in the embodiments of the present application more clearly, a brief description will be given below of the drawings necessary for the description of the embodiments; apparently, the drawings in the description below are only some embodiments of the present application, and a person of ordinary skill in the art can obtain other drawings based on these drawings without involving any inventive effort.

FIG. 1 is a schematic diagram showing a structure of an electric cutter assembly according to Embodiment 1 of the present application;

FIG. 2 is a schematic diagram showing a partial structure of the electric cutter assembly according to Embodiment 1 of the present application;

FIG. 3 is a schematic diagram showing a structure of a base of the electric cutter assembly according to Embodiment 1 of the present application;

FIG. 4 is a schematic diagram showing a partial structure of the base according to Embodiment 1 of the present application;

FIG. 5 is a cross-sectional view of a portion of the base of the electric cutter assembly according to Embodiment 1 of the present application;

FIG. 6 is an enlarged view at A in FIG. 5;

FIG. 7 is a schematic diagram showing a structure of the sheath component in the electric cutter assembly according to Embodiment 1 of the present application;

FIG. 8 is a schematic view showing a structure of a blade component in the electric cutter assembly according to Embodiment 1 of the present application;

FIG. 9 is a schematic diagram showing a partial structure of the sheath component in the electric cutter assembly according to Embodiment 1 of the present application;

FIG. 10 is a cross-sectional view of a portion of the sheath component in the electric cutter assembly according to Embodiment 1 of the present application;

FIG. 11 is a schematic diagram showing a structure of a handle component in the electric cutter assembly according to Embodiment 1 of the present application;

FIG. 12 is a schematic diagram showing the structure of the handle component in the electric cutter assembly according to Embodiment 1 of the present application from another perspective;

FIG. 13 is a schematic diagram showing a partial structure of the handle component according to Embodiment 1 of the present application;

FIG. 14 is a front view of a portion of the handle component in the electric cutter assembly according to Embodiment 1 of the present application;

FIG. 15 is a schematic diagram showing a structure of a portion of the handle component in the electric cutter assembly according to Embodiment 1 of the present application;

FIG. 16 is a schematic diagram showing the structure of the portion of the handle component in the electric tool kit according to Embodiment 1 of the present application from another perspective;

FIG. 17 is a schematic diagram showing a structure of a driving component in the electric cutter assembly according to Embodiment 1 of the present application;

FIG. 18 is a side view of the driving component in the electric cutter assembly according to Embodiment 1 of the present application;

FIG. 19 is a schematic diagram showing structures of a bracket, a safety component, a switch component, and a locking component in the electric cutter assembly according to Embodiment 1 of the present application;

FIG. 20 is a side view of the bracket, the safety component, the switch component, and the locking component in the electric cutter assembly according to Embodiment 1 of the present application.

### DETAILED DESCRIPTION OF THE INVENTION

To describe the technical aspects, the object, and the effects of the present application in detail, the following description is made in conjunction with the embodiments and the accompanying drawings.

### Embodiment 1

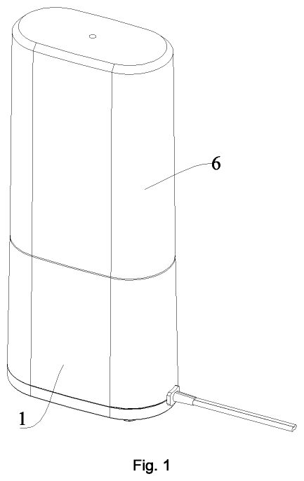

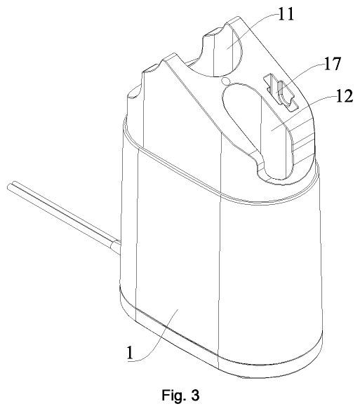

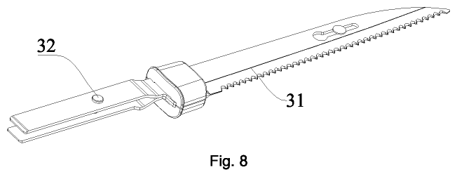

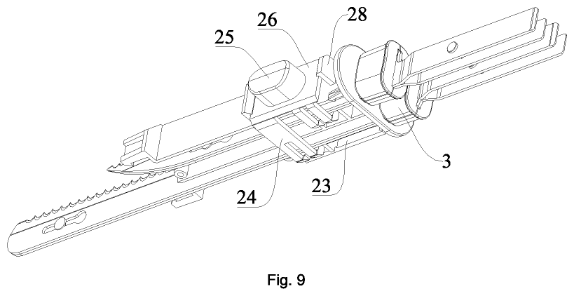

With reference to FIGS. 1 to 20, Embodiment 1 of the present application discloses an electric cutter assembly for cutting bread, meat, bones, and other food materials and facilitating a user's operation in processing various food materials.

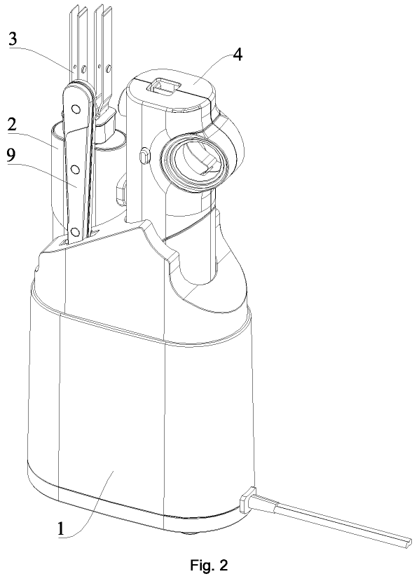

As shown in FIGS. 1 and 2, the electric cutter assembly includes a base 1 and a cover 6, wherein the base 1 is provided with a detachable handle component 4 and a sheath component 2, the sheath component 2 receives two blade components 3 which can be taken out from the sheath component 2, and the handle component 4 can form an electric cutter with any of these blade components 3; the blade component 3 mounted to the handle component 4 can reciprocate with respect to the handle component 4 under the drive of the handle component 4, and thus the user can complete the cutting of the food materials by moving the blade component 3 while holding the handle component 4.

With reference to FIGS. 2, 3 and 4, the base 1 is provided with a first chamber 11 for receiving the sheath component 2 and a second chamber 12 for receiving the handle component 4, the sheath component 2 has a first housing 21 mating with the first chamber 11, the handle component 4 has a second housing 41 mating with the second chamber 12, and the base 1 and the cover 6 cooperate to enclose a closed chamber for receiving the sheath component 2, the handle component 4, and the blade component 3, which facilitates the storage of the electric cutter while reducing the influence of the external environment on the electric cutter.

In detail, a storage box 8 is further provided in the base 1, and the storage box 8 has a third chamber 81; the base 1 is provided with a limiting recess 17 which is in communication with an opening of the storage box 8; tableware such as forks 9 and chopsticks can be placed in the storage box 8, and once the tableware such as forks 9 is placed in the storage box 8, the storage box 8 is engaged with the limiting recess 17, to prevent the tableware from falling down in the storage box 8, which is convenient for the user to access.

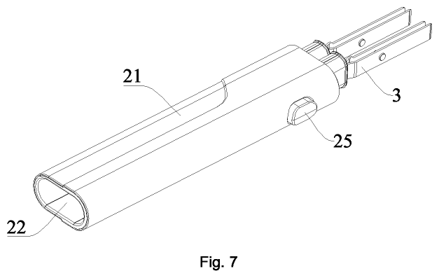

As shown in FIGS. 7, 8 and 9, the blade component 3 includes two blades 31 arranged in parallel, the shape and model of the blades 31 can be configured as required, the sheath component 2 includes a fixed seat 23 for receiving the blades 31 and a movable seat 24 movable relative to the fixed seat 23, the movable seat 24 presses the blades 31 against the fixed seat 23, the movable seat 24 is provided with a movable button 25 for driving the movable seat 24 to move relative to the fixed seat 23, the first housing 21 surrounds the fixed seat 23, the movable seat 24 and a portion of the blade 31 within the fixed seat 23 secure a sharpened portion of the blade 31 within the sheath component 2 to prevent the user from getting hurt when the user accesses the blade component 3, and the movable button 25 protrudes out of the first housing 21 to facilitate taking out the blade component 3 from the sheath component 2 by the user.

Specifically, the movable seat 24 has a movable plate 26 and the movable button 25 is fixedly connected to the movable plate 26; the movable plate 26 is provided with an engaging block 29 connected to the movable plate 26; the engaging block 29 abuts against the blade 31 to press the blade component 3 against the fixed seat 23; an elastic member 27 is provided between the movable plate 26 and one of the sidewalls of the fixed seat 23. A telescopic direction of the elastic member 27 coincides with a movement direction of the movable plate 26, and the elastic member 27 drives the movable plate 26 to move in a direction away from the fixed seat 23; a clasp 28 for clasping the movable plate 26 is provided on the fixed seat 23 to limit a movement range of the movable seat 24 relative to the fixed seat 23 and prevent the movable seat 24 from falling off.

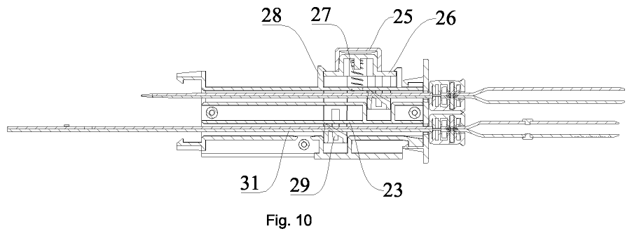

With reference to FIG. 10, when the elastic member 27 is in a free state, the engaging block 29 is held to abut against the blade 31 under the action of the elastic member 27, at this time, the blade component 3 is locked in the sheath component 2; when it is necessary to remove the blade component 3, the movable button 25 is pressed to enable the movable plate 26 to get closer to the fixed seat 23, and the engaging block 29 is driven by the movable plate 26 to move in a direction away from the blade 31 to release the blade component 3 so that the blade component 3 can be pulled out from the fixed seat 23. Meantime, the elastic member 27 is compressed, and after the blade component 3 is taken out and the movable button 25 is released, the movable button 25 automatically resets under the action of the elastic member 27, and by the same reasoning, when it is necessary to place the blade component 3 in the sheath component 2, it would be enough by pressing the movable button 25 to operate the sheath component 2.

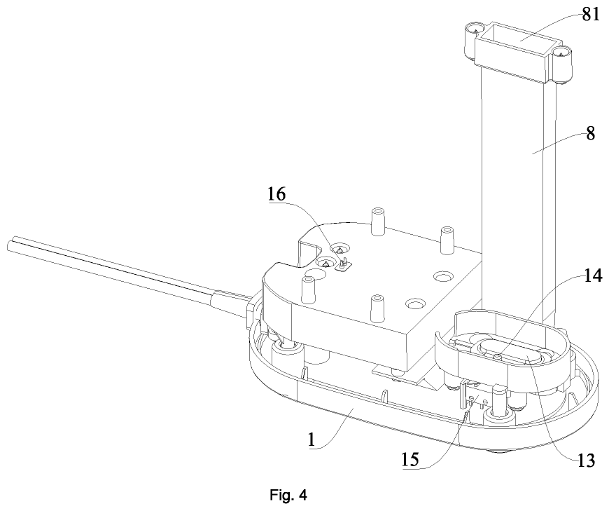

As shown in FIGS. 4 and 7, the electric cutter assembly further includes an electric cutter disinfection device, wherein a bottom of the first housing 21 has a through-hole 22 penetrating the first housing 21, the first chamber 11 is provided with a UV lamp 13, and the light emitted from the UV lamp 13 is directed to the through-hole 22; after the first housing 21 is placed into the first chamber 11, the UV lamp 13 is activated to disinfect the blade 31 by means of the ultraviolet rays emitted by the UV lamp 13, kill bacteria on the blade 31, and improve the sanitation degree of the electric cutter assembly.

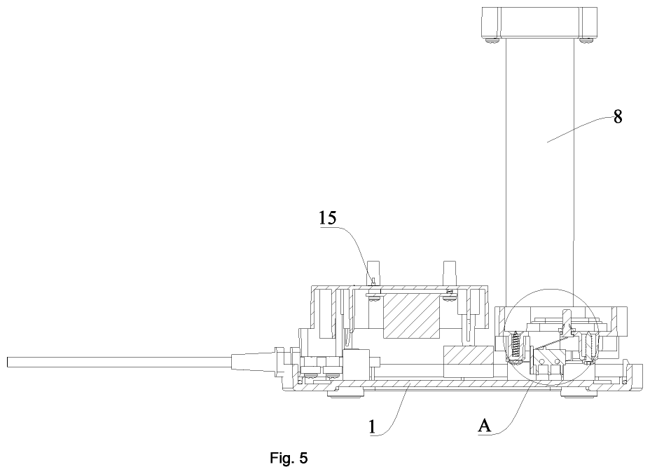

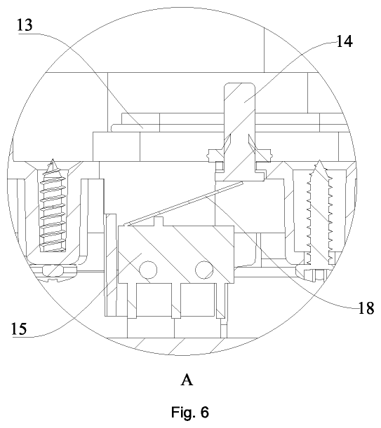

With reference to FIGS. 5 and 6, the electric cutter disinfection device further includes a triggering component, wherein the trigger component includes a triggering lever 14 provided on the base 1 and movable in a direction of the first housing 21 entering and exiting the first chamber 11, and one end of the triggering lever 14 extends into the first chamber 11. The triggering component further includes a second switch 15 electrically connected to the UV lamp 13, and the second switch 15 is positioned at an end of the triggering lever 14 away from the first chamber 11; the second switch 15 has a shrapnel 18 and the shrapnel 18 abuts against an end of the triggering lever 14 away from the first chamber 11. In the process of placing the sheath component 2 into the first chamber 11, the first housing 21 abuts against the triggering lever 14 and drives the triggering lever 14 to move in a direction closer to the second switch 15, and after the sheath component 2 is completely placed into the first chamber 11, the triggering lever 14 abuts against the shrapnel 18 on the second switch 15 and turns on the second switch 15, and at this time, the UV lamp 13 starts disinfecting the blade 31; when the sheath component 2 is removed from the first chamber 11, the triggering lever 14 is reset by actuation of the shrapnel 18, and when the triggering lever 14 is reset, the second switch 15 is turned off, and the UV lamp 13 is turned off.

Furthermore, the blade 31 can be automatically disinfected by placing the sheath component 2 on the base 1, without active operation by the user, and the blade 31 can be kept clean while the user operation is facilitated.

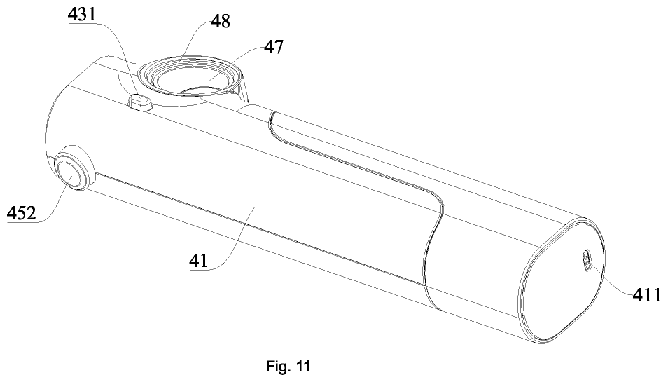

As shown in FIGS. 11 to 16, the handle component 4 further includes a bracket 42 provided in the second housing 41, wherein the bracket 42 is provided with a safety component 43, a switch component 44, a locking component 45, and a driving component 46, and the second housing 41 is provided with an insert hole 412 for the blade 31 to be partially inserted into the driving component 46, facilitating assembling the blade component 3 and the handle component 4.

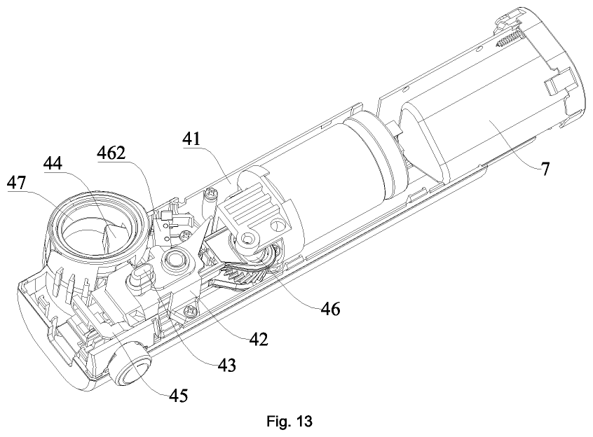

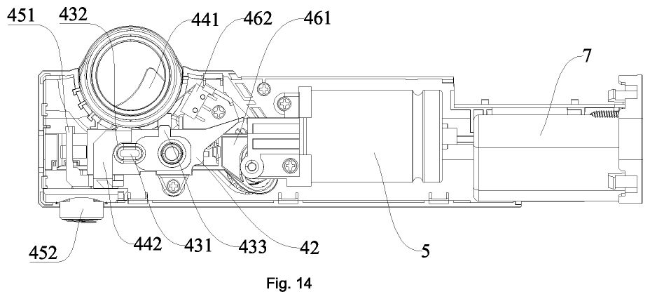

With reference to FIGS. 13 and 14, the driving component 46 includes a mount 461 movable relative to the bracket 42, a driving member 5 for driving the movement of the mount 461, and a first switch 462 electrically connected to the driving member 5; the mount 461 has a receiving groove 463 for receiving a part of the blade 31, an opening of the receiving groove 463 is aligned with the insert hole 412, and the blade component 3 can be assembled to the handle component 4 by means of the cooperation of the blade 31 and the receiving groove 463; furthermore, the driving member 5 drives the mount 461 to reciprocate with respect to the bracket 42, thereby driving the cutter component to reciprocate with respect to the handle component 4.

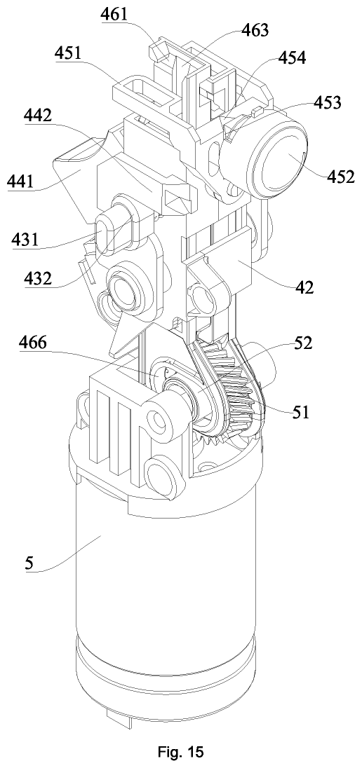

As shown in FIGS. 13 and 15, the locking component 45 includes clamping portions 451 rotatably provided on the bracket 42, two clamping portions 451 are provided, and the two clamping portions 451 extend, respectively, into the receiving grooves 463 from opposite sides of the mount 461 to fix the position of the blade component 3 relative to the handle component 4 by the two clamping portions 451 clamping the blade 31, and the locking component 45 further includes an unlocking button 452 driving the clamping portion 451 to rotate in a direction away from the blade component 3, and the blade component 3 can be released by pressing the unlocking button 452.

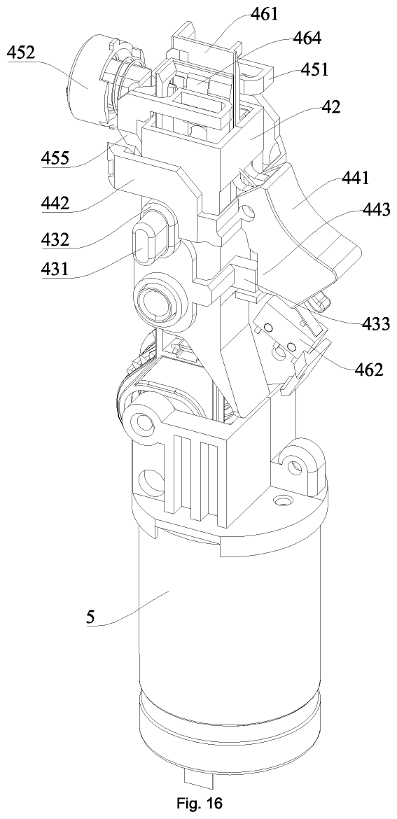

With reference to FIGS. 14 and 16, the switch component 44 includes a trigger 441, wherein the trigger 441 is rotatably configured on the bracket 42, and the trigger 441 has two limiting portions 442 arranged at intervals along a length direction of a rotational axis of the trigger 441, and when the trigger 441 rotates, the limiting portions 442 can be driven to move in a direction closer to the clamping portion 451. Along with the movement of the limiting portion 442, the limiting portions 442 abut against the clamping portions 451 in a one-to-one correspondence to drive the clamping portion 451 to rotate in a direction closer to the blade component 3 until the clamping portion 451 abuts against the blade 31, then the two limiting portions 442 remain respectively abutted against the two clamping portions 451, and at this time, the limiting portions 442 prevent the clamping portions 451 from rotating in a direction away from the blade component 3. Even if the unlocking button 452 is pressed, the limiting portion 442 cannot be rotated in a direction away from the blade component 3 to release the blade component 3.

In detail, the trigger 441 further includes a triggering portion 443, when the trigger 441 is pressed to rotate, the triggering portion 443 moves in a direction closer to the first switch 462 and abuts against the first switch 462 to turn on the first switch 462, and at this time, the driving member 5 is activated and drives the mount 461 to act, and when the trigger 441 is reset, the triggering portion 443 is disengaged from the first switch 462 so that the first switch 462 is turned off, and then the driving member 5 is also stopped.

As shown in FIGS. 14 and 15, the safety component 43 includes two safety buttons 431, wherein the two safety buttons 431 are respectively mounted on two opposite sides of the bracket 42, the two safety buttons 431 are respectively movable along a length direction of a rotational axis of the trigger 441, and the two safety buttons 431 respectively have a first locking portion 432, and when the trigger 441 is in a state where the first switch 462 is turned off, the two first locking portions 432 abut against the two limiting portions 442 in one-to-one correspondence, the rotation generated when the trigger 441 is pressed is blocked by the first locking portion 432, hence the first locking portion 432 should be misaligned with the limiting portion 442 by pressing the safety button 431 before the first switch 462 is turned on so that the trigger 441 can be successfully rotated.

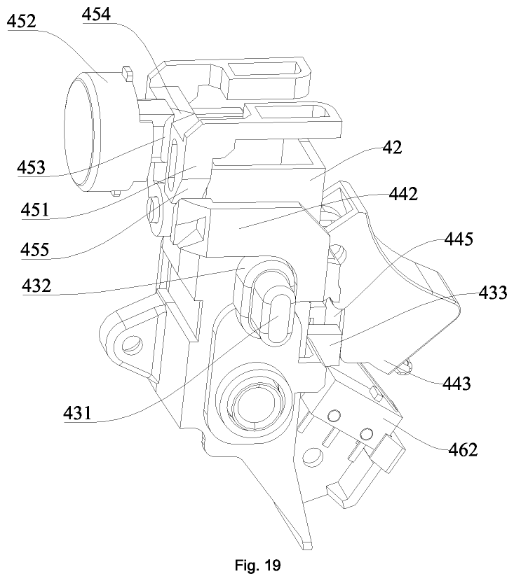

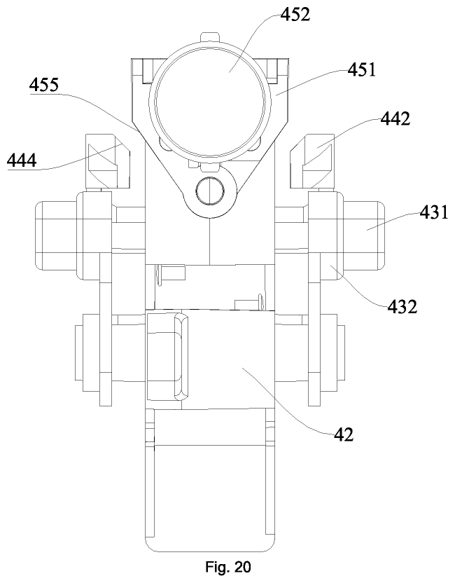

As shown in FIGS. 19 and 20, when using the electric cutter, the user firstly takes out the blade component 3 from the sheath component 2 and assembles same onto the handle component 4; since the first locking portion 432 and the limiting portion 442 abut against each other, the trigger 441 cannot be pressed, the electric cutter cannot be directly started at this moment; the user has to press a safety catch so that the first locking portion 432 and the limiting portion 442 are misaligned before pressing the trigger 441 to start the driving member 5, whereby the mount 461 drives the blade component 3 to move under the action of the driving member 5. The user presses the trigger 441 while the limiting portion 442 rotates and drives the clamping portion 451 to rotate in a direction closer to the blade component 3, to clamp the blade from opposite sides of the blade component 3 by means of two clamping portions 451 respectively and fix the blade component 3 on the mount 461. The position of the blade component 3 is locked while the electric cutter is activated, and after the electric cutter is activated, the two limiting portions 442 respectively abut against the two clamping portions 451 to limit the rotation of the clamping portion 451 in a direction away from the blade component 3 by means of the limiting portions 442, hence the user cannot release the blade component 3 from the clamping portion 451 even if the unlocking button 452 is touched by mistake during use of the electric cutter. When the user removes the blade component 3, the trigger 441 needs to be pressed first to reset the trigger 441, at this time, the triggering portion 443 is separated from the first switch 462 to stop the operation of the driving member 5, the trigger 441 is reset while the limiting portion 442 rotates and moves away from the clamping portion 451, the clamping portion 451 is no longer limited by the limiting portion 442, and the user can press the unlocking button 452 to drive the clamping portion 451 to rotate in a direction away from the blade component 3 to unlock the blade component 3, and then the blade component 3 can be withdrawn from the mount 461.

With reference to FIGS. 15 and 19, one end of the unlocking button 452 close to the clamping portion 451 has a first inclined surface 453, and the clamping portion 451 is provided with a second inclined surface 454 mating with the first inclined surface 453; when the unlocking button 452 is pressed, the first inclined surface 453 and the second inclined surface 454 abut against and slide relative to each other as the unlocking button 452 moves, so that the clamping portion 451 smoothly rotates in a direction away from the blade component 3 under the action of the unlocking button 452, and the user is facilitated to unlock the blade component 3 by operating the unlocking button 452.

As shown in FIGS. 16 and 20, one side of the limiting portion 442 close to the clamping portion 451 has a third inclined surface 444, and the clamping portion 451 is provided with a fourth inclined surface 455 mating with the third inclined surface 444; when the limiting portion 442 abuts against the clamping portion 451 to fix the position of the clamping portion 451, the third inclined surface 444 abuts against the fourth inclined surface 455 to limit the rotation of the clamping portion 451 in a direction away from the blade component 3, locking the position of the clamping portion 451, and ensuring that the relative positions of the two clamping portions 451 and the two limiting portions 442 keep unchanged.

Specifically, a second locking portion 433 is further provided on the safety button 431, and the second locking portions 433 on the two safety buttons 431 respectively abut against the triggering portion 443 to prevent the triggering portion 443 from turning on the first switch 462 when the user accidentally touches the trigger 441. Moreover, the triggering portion 443 is provided with a locking notch 445 for receiving the second locking portion 433, after the user presses the two safety buttons 431, the second locking portion 433 is dislocated from the triggering portion 443 and the two second locking portions 433 are attached to each other; after pressing the trigger 441, both of the two second locking portions 433 enter the locking notch 445, to limit the safety button 431 by means of the locking notch 445 during the operation of the electric cutter, preventing the safety button 431 from an unexpected action.

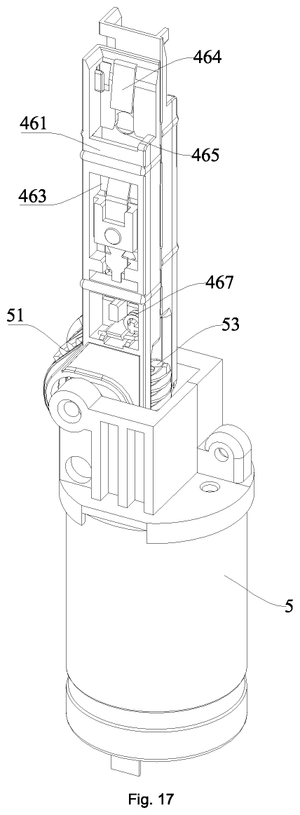

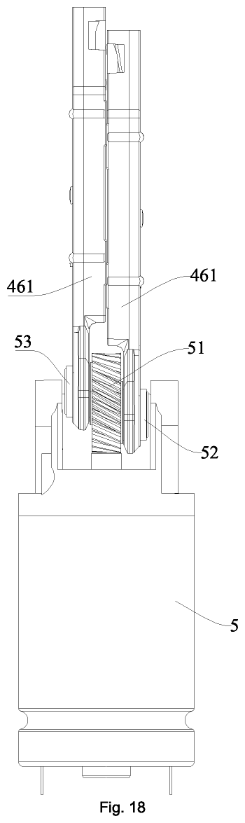

With reference to FIGS. 17 and 18, in this embodiment, two mounts 461 are provided on the bracket 42, the two mounts 461 are arranged in parallel, and the two mounts 461 are respectively mated with the two blades 31 of one blade component 3 in a one-to-one correspondence, sides of the two mounts 461 away from each other are respectively provided with the receiving groove 463, and a locking shrapnel 464 for pressing the blades 31 into the receiving groove 463 is provided in the receiving groove 463; once the blade 31 is inserted into a gap between the locking shrapnel 464 and the receiving groove 463, the blade 31 can be pressed against the mount 461 by means of the locking shrapnel 464 to position the blade component 3 before the clamping portion locks the blade component 3, and prevent the blade 31 from falling off from the mount 461 during the operation of the safety component 43, the switch component 44, and the locking component 45.

In detail, the blade 31 is provided with a protruding limiting post 32, the locking shrapnel 464 is provided with a limiting hole 465 mating with the limiting post 32, and after the blade 31 is inserted into the gap between the locking shrapnel 464 and the mount 461, the limiting post 32 extends into the limiting hole 465 to position the blade component 3. A torsion spring 467 is further provided in the receiving groove 463, wherein the torsion spring 467 abuts against an end of the blade 31 extending into the receiving groove 463, and the torsion spring 467 can limit a depth of the blade 31 inserted into the receiving groove 463 to ensure that the limiting post 32 is aligned with the limiting hole 465, and apply an ejection force to the blade 31 when the user pulls out the blade component 3 so that the limiting post 32 can smoothly exit the limiting hole 465 when the blade component 3 is pulled by the user, and it is easier to disassemble the blade component 3.

Alternatively, when the user disassembles the blade component 3, the sheath component 2 can be first sheathed onto the blade component 3, and then the unlocking button 452 can be pressed, and the blade component 3 together with the sheath component 2 can be removed from the handle component 4, effectively avoiding direct contact of the user's hand with the blade component 3 and preventing the user from getting hurt when disassembling the blade component 3.

With reference to FIGS. 15 and 18, in this embodiment, the driving component 46 further includes a transmission gear 51 and a worm 53 meshing with the transmission gear 51, wherein the worm 53 mates with an output end of the driving member 5 to drive the worm 53 to rotate under the action of the driving member 5, and further drives the transmission gear 51 to rotate. A protruding transfer link 52 is respectively provided on either of the opposite side surfaces of the transmission gear 51, and the two transfer links 52 are respectively arranged eccentrically with respect to a rotational axis of the transmission gear 51, and axial directions of the two transfer links 52 are not in one straight line. One end of the mount 461 close to the transmission gear 51 is provided with an oblong hole 466, the transmission gear 51 is located between the two mounts 461, and two transmission shafts respectively extend into the oblong hole 466 in their vicinity, and the transmission shaft abuts against an inner peripheral wall of the oblong hole 466, hence during the rotation of the transmission gear 51, the transmission shaft will generate a reciprocating motion in a vertical direction in FIG. 18, and thus the mount 461 moves synchronously with the transmission shaft, that is, the blade 31 component is enabled to reciprocate relative to the handle component 4 to cut the food materials.

As shown in FIGS. 4, 11 and 14, a storage battery 7 electrically connected to the driving member 5 is further provided in the second housing 41, the driving member 5 is driven to move because the storage battery 7 supplies electric power to the driving member 5; a charging interface 411 electrically connected to the storage battery 7 is provided on the second housing 41, a charging plug 16 is provided on the base 1, and the charging plug 16 is located at a bottom of the second chamber 12; after the handle component 4 is placed in the second chamber 12, the charging plug 16 interfaces with the charging interface 411, and then the storage battery 7 can be charged, or the battery 7 can be charged by directly connecting the storage battery 7 with the charging interface 411 using a charging cord, to ensure that the storage battery 7 is fully charged when the user uses the electric cutter.

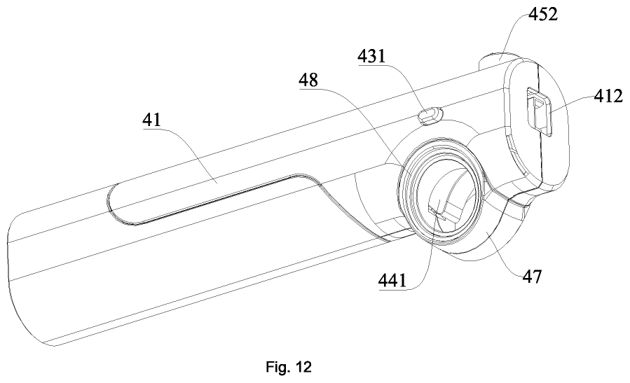

With reference to FIGS. 11 and 12, the second housing 41 encloses the bracket 42 and the parts provided on the bracket 42 to prevent foreign objects entering the handle component 4, and the second housing 41 is further provided with a via-hole for exposing the unlocking button 452, the trigger 441, and the safety button 431, to facilitate the operation of the handle component 4 for the user. To prevent the user from touching the trigger 441 by mistake, the second housing 41 is further provided with a protection ring 47 around the trigger 441, the trigger 441 is received in an area surrounded by the protection ring 47, and the protection ring 47 is provided with an indicating lamp 48 electrically connected to the first switch 462, and when the user presses the trigger 441 to turn on the first switch 462, the indicating lamp 48 lights up, hence the user can visually know the operation state of the handle component 4 and avoid the user's accidental operation.

In summary, the electric cutter assembly provided in the present application is powered by a built-in storage battery, which effectively avoids the hindrance of a power cord for a user, and the storage battery can be charged through a power cord or a base, hence it is easier for the user to operate; the electric cutter assembly has a chamber enclosed by the base and the cover for receiving tools such as the handle component, the sheath component, and forks, wherein the sheath component receives a plurality of blade components, and the user can combine the blade component to be used with the handle component as needed, and the blade component can be driven to reciprocate with respect to the handle component under the action of the handle component so as to cut the food materials; the handle component in the electric cutter assembly automatically locks the blade component when the handle component starts through the cooperation of the safety component, the switch component, the locking component, and the driving component, preventing the blade component falling off during use in case that the user forgets to lock the blade component; further, the cooperation between the limiting portion and the clamping portion during the operation of the electric cutter ensures that the user cannot release the blade component when mistakenly touching the unlocking button and get hurt, hence the electric cutter is more safe and reliable; moreover, the electric cutter has the UV lamp to be triggered by the first housing of the sheath component within the base, and the blade in the sheath component can be automatically disinfected and sterilized by placing the sheath component into the first chamber of the base, whereby the hygienic degree of the electric cutter is improved.

The above-mentioned embodiment is merely an example of the present application and is not intended to limit the scope of the present application, and all changes that come within the meaning and range of equivalency of the present application and the appended claims are to be embraced within the scope thereof.

What is claimed is:

1. An electric cutter assembly, comprising a handle component and a blade component configured movably on the handle component, wherein the handle component is provided therein with a driving component for driving the blade component to reciprocate relative to the handle component and a storage battery electrically connected to the driving component, and the handle component is further provided with a charging interface electrically connected to the storage battery.

2. The electric cutter assembly according to claim 1, further comprising a charging cord adapted to the charging interface.

3. The electric cutter assembly according to claim 1, further comprising a base having a second chamber for receiving the handle component, the second chamber having a charging plug for mating with the charging interface.

4. The electric cutter assembly according to claim 3, further comprising a sheath component having at least one removable blade component received therein, the base having a first chamber for receiving the sheath component.

5. The electric cutter assembly according to claim 4, wherein the sheath component has a first housing for receiving the blade component, a through hole is provided in a bottom surface of the first housing to penetrate the first housing, a UV lamp is provided in the first chamber, and light emitted from the UV lamp is directed to the through hole.

6. The electric cutter assembly according to claim 5, wherein a movable triggering lever is provided on the base and an end of the triggering lever extends into the first chamber, a second switch electrically connected to the UV lamp is further provided on the base, and the second switch is configured near an end of the triggering lever away from the first chamber.

7. The electric cutter assembly according to claim 6, wherein the second switch comprises a shrapnel that abuts against the end of the triggering lever away from the first chamber.

8. The electric cutter assembly according to claim 5, wherein the first housing is provided therein with a fixed seat for receiving the blade component and a movable seat pressing the blade component against the fixed seat, the movable seat is provided with a movable button for driving the movable seat to move, and the movable button protrudes from the first housing.

9. The electric cutter assembly according to claim 8, wherein the movable seat has a movable plate to which the movable button is mounted, an elastic member for driving the movable plate to move away from the fixed seat is provided between the movable plate and the fixed seat, and a clasp is provided on the fixed seat to mate with the movable plate.

10. The electric cutter assembly according to claim 9, wherein an engaging block is provided on the movable plate, and the engaging block is inserted into the fixed seat and moved towards the fixed seat by the elastic member.

11. The electric cutter assembly according to claim 1, wherein the handle component has a second housing and a bracket is provided in the second housing, the bracket is provided with a safety component, a switch component, a locking component and a driving component, the driving component comprises a mount movable relative to the bracket and a driving member for driving the mount to reciprocate, and the mount mates with the blade component; the locking component comprises a rotatable clamping portion for pressing the blade component against the mount, and the locking component further comprises an unlocking button to drive the clamping portion to rotate in a direction away from the mount; the switch component comprises a trigger configured rotatably on the bracket, and the trigger has a limiting portion for driving the clamping portion to rotate in a direction closer to the mount; the safety component has a first locking portion that abuts against the limiting portion to limit rotation of the trigger.

12. The electric cutter assembly according to claim 11, wherein the driving component further comprises a first switch electrically connected to the driving member, the trigger has a triggering portion that mates with the first switch, and the limiting portion abuts against the clamping portion when the triggering portion contacts the first switch.

13. The electric cutter assembly according to claim 12, wherein a second locking portion abutting against the triggering portion is also provided on the safety component, and the trigger is provided with a locking notch for receiving the second locking portion.

14. The electric cutter assembly according to claim 11, wherein the mount has a receiving groove for receiving a portion of the blade component, the clamping portion extending into the receiving groove for abutting against the blade component.

15. The electric cutter assembly according to claim 14, wherein the blade component comprises two blades arranged side by side, two mounts are provided, and the two mounts are arranged side by side, surfaces of the two mounts away from each other are provided with the receiving grooves, respectively, and one of the receiving grooves receives one of the blades.

16. The electric cutter assembly according to claim 15, wherein a locking shrapnel is provided within the receiving groove to abut against the blade and press the blade against the mount.

17. The electric cutter assembly according to claim 16, wherein the blade is provided with a protruding limiting post, and a limiting hole mating with the limiting post is provided in the locking shrapnel.

18. The electric cutter assembly according to claim 11, wherein one end of the mount close to the driving member has an oblong hole, an output end of the driving member is provided with a transmission gear, a protruding transfer link is provided on a sidewall of the transmission gear, the transfer link is eccentrical with respect to a rotational axis of the transmission gear, and the transfer link extends into the oblong hole and abuts against an inner peripheral wall of the oblong hole.

19. The electric cutter assembly according to claim 11, wherein an end of the unlocking button close to the clamping portion has a first inclined surface, and the clamping portion is provided with a second inclined surface mating with the first inclined surface.

20. The electric cutter assembly according to claim 11, wherein a side of the limiting portion close to the clamping portion has a third inclined surface, and the clamping portion is provided with a fourth inclined surface mating with the third inclined surface.
